(19) United States
(12) Patent Application Publication
(10) Pub. No.: US 2023/0033963 A1
(43) Pub. Date: Feb. 2, 2023

(54) Multiple Physiological Data Collection and Analysis Device and System

(71) Applicant: LARGAN Health AI-Tech Co., LTD., Taipei City (TW)

(72) Inventors: Chen Hsiang CHEN, New Taipei City (TW); Cheng Kuo LAI, Taipei City (TW)

(21) Appl. No.: 17/491,589

(22) Filed: Oct. 1, 2021

(30) Foreign Application Priority Data
Jul. 26, 2021 (TW) 110127399

(51) Int. Cl. G16H 50/20; G16H 40/63

(52) U.S. Cl. CPC G16H 50/20; G16H 40/63

(57) ABSTRACT

Disclosed are collection and analysis devices and systems for multiple types of physiological data that collect, manually mark, sampling physiological data for machine learning and AI analyses, all in a single operational background. Physiological data gathered/uploaded by sensing devices of different type and function, described in different formation, recorded at different times and/or pertaining to different patients are processed in one system, to discover and evaluate correlation between a type of physiological data and a certain physiological phenomenon.

### FIELD OF THE INVENTION

The present invention relates to a physiological data analysis device and system, in particular to a device and system to collect, process and display physiological data of multiple types, gathered by sensing devices of different types or function and defined in various forms and descriptions, in order to find a correlation of a type of physiological data and specific physiological phenomenon.

### BACKGROUNDS OF THE INVENTION

Polysomnography (PSG) is the most commonly used standard inspection method in sleep medicine and the diagnosis of sleep related diseases such as sleep disorders, snoring, epilepsy, and sleep apnea. The inspection is usually carried out in a hospital ward. The patient must stay in the hospital, usually in the sleep center, and the doctor or sleep technician installs a variety of sensors on the patient to gather the sleep related physiological data throughout the night. The inspection results are displayed at intervals of, for example, every 30 seconds. Taking a 6-hour inspection as an example, 720 units of inspection results will be produced, which are then processed and provided to the doctor for diagnosis.

PSG needs the combination of multiple instruments to complete the inspection and provide for comprehensive diagnosis. Inspection items usually include:
1. Electroencephalography (EEG): The readings and their changes of EEG in various stages of sleep (N1, N2, N3, and REM).
2. Electromyography (EMG): Including mandibular electromyogram, to help determining the sleep stage, and leg electromyography, to find abnormal leg twitches.
3. Electrocardiography (ECG): Changes in heart rate during sleep, which are also useful in finding arrhythmia problems.
4. Electrooculogram (EOG): Useful in the judgment of rapid eye movement.
5. Blood oxygen saturation (SaO2) and pulse: The state of blood oxygen concentration and pulse changes.
6. Tho/Abdo Effort: The breathing situation.
7. Nasal-Oral Air Flow: The ventilation status of the upper respiratory tract.

As there are too many items to be inspected by PSG, the multiple inspection instruments attached to the patient do not only affects the patient's sleep, but also lead to inaccurate detection. In addition, the statistics and marking of the results are also quite labor-intensive. To solve this technical problem, the industry has proposed a variety of solutions that performs fewer types of inspection items, supplemented by software, to automatically mark inspection results. For example, for the diagnosis of sleep apnea, a simplified sleep physiology examination device was developed. The device only needs to measure nasal airflow, pulse, and blood oxygen concentration. The collected data can be interpreted by a machine to generate a sleep apnea test result similar to PSG, namely the sleep apnea index (Apnea-Hypopnea Index, AHI).

A paper by Sun et al. found out, after deep learning with a large amount of PSG data, that adding the value of abdominal tension to the ECG signal, it is possible to calculate a sleep staging result that is quite close to diagnosis results using brain waves. See Haoqi Sun et al., “Sleep Staging from Electrocardiography and Respiration with Deep Learning.” Dec. 21, 2019, Sleep 2020, https://academic.oup.com/sleep/article-abstract/43/7/zsz306/5682785.

Largan Health AI-Tech also performed machine learning on a large amount of PSG data and announced a sleep analysis software that uses ECG signals, only, and provides diagnosis results quite close to the sleep staging and apnea index by using PSG.

With the popularization of wearable devices, IoT sensing technology, and millimeter wave technology, many experts try to place more instruments on the subject, hoping to more accurately detect and predict certain physiological phenomena, and/or find out the cause, seek ways to improve health. However, instruments or measurement methods that are useful for the detection, prediction, or cause analysis of physiological phenomena have not been discovered because of the new technologies and new products. The accuracy of the measurement has not been improved accordingly, either.

### SUMMARY OF THE INVENTION

The purpose of the present invention is to provide a novel multiple physiological data collection and analysis device, as a solution for multiple physiological data collection, manual marking, machine learning, training sampling, AI analysis and other processes, all in a single environment.

The objective of this invention is to provide a tool that is convenient for professionals to quickly find in the vast sea of data the types of physiological data that are correlated to specific physiological phenomena.

The present invention provides a multiple physiological data collection and analysis device that obtains/receives physiological data from various sensing devices and automatically classifies and stores the physiological data. After machine learning, certain types of physiological data correlated to specific physiological phenomena can be found and evaluated.

The present invention provides a multiple physiological data analysis system that can display different types of physiological data received from various sensing devices on the same display device according to the conditions set by the user. The present invention provides a convenient tool for researchers to discover a correlation of a type of physiological data and certain physiological phenomena.

The present invention provides a multiple physiological data collection and analysis device that provides useful evaluation tools to determine the correlation between specific types of physiological data and specific physiological phenomena.

To achieve the above objectives, the present invention provides a multiple physiological data collection and analysis device, which comprises:

a data upload device to provide a communication channel for communication link of a plurality of physiological data sensing devices or physiological data storage devices, to receive different types of physiological data from the plurality of physiological data sensing devices or physiological data storage devices;

a data storage device to provide a large memory space for storing various physiological data and result data of the physiological data processed in the multiple physiological data collection and analysis device;

a data editing device to provide a human-machine interface for users to retrieve specific types of physiological data and/or result data from the data storage device, and for browsing or manually adding, deleting or modifying a marker on a set of the physiological data, and for selecting a type of physiological data entry for evaluation of a correlation with a marker;

wherein the data storage device provides automatic indexing capability, to automatically index a set of physiological data and/or result data, and wherein the data editing device is configured to display physiological data in an arrangement according to a corresponding index in response to an input request; and

a correlation evaluation device to calculate a correlation value of a type of physiological data and a marker.

The multiple physiological data collection and analysis device of the present invention may further comprise an automatic analysis device that provides a filtering interface to receive a filtering instruction and to automatically retrieve corresponding physiological data and/or result data from the data storage device, and to discover from the multiple physiological data a type of physiological data that is correlated to a specific marker.

In a preferred embodiment of the present invention, the physiological data stored in the data storage device correspond to a plurality of person and are classified into four categories: “signal-featured” physiological data, “multi-lead signal-featured” physiological data “frame-featured” physiological data and “multiple frame-featured” physiological data. Each set of physiological data is indexed with the following features:

For “signal-featured” physiological data and “multi-lead signal-featured” physiological data: file name, recording time and an identification code (ID code).

For “frame-featured” physiological data and “multiple frame-featured” physiological data: file name, recording time and an identification code (ID code).

Among these features, the file name preferably includes a personal ID code of the person from whom the physiological data set was gathered. The recording time can include a time point or a time period defined by a start time and an end time. As for the ID code, it is preferably a unique code and is preferably related to the type of physiological data included in the corresponding data set. The code length should be moderate, that is, it should not be too short to easily repeat with the ID code of another person or data set, and it should not be too long, which increases processing complexity, resources and time. In the preferred embodiments of the present invention, the ID code may comprise a hash value, especially the “Secure Hash Algorithm 256-bit” (SHA256) value, calculated according to the numerical value of the physiological data of a corresponding data set.

Mainly because of the unique data and information classification methods and specially designed indexing methods of the present invention, data in different forms, with different properties, in different storage or transmission media, and with different data volumes, data relating to different people and recorded at different times, can all be stored in a single storage device and can be retrieved, filtered, edited and otherwise utilized using a single display interface or human-machine interface, whereby possible correlations among a plurality of data set can be immediately shown or revealed. In addition, the possible correlations between various types of physiological data and the markers attached to a set of physiological phenomena can be easily discovered from the display interface or easily recognized by the invented analysis system. The present invention is useful for skilled persons to discover a type of physiological data that may be a controlling factor or key factor of a physiological phenomenon but is yet known to the world.

Other objectives and advantages of the present invention will become more apparent from the following detailed description with reference to the accompanying drawings.

### BRIEF DESCRIPTION OF THE DRAWINGS

FIG. 1 shows a schematic diagram of an embodiment of the multiple physiological data collection and analysis device of the present invention.

FIG. 2 shows archive formats of several examples of “signal-featured” physiological data usable in the present invention.

FIG. 3 shows an example of archive format of a “frame-featured” of physiological data usable in the present invention.

FIG. 4 shows a schematic diagram of a data structure for storing physiological data in the data storage device of the multiple physiological data collection and analysis device of the present invention.

FIG. 5 shows a flow chart of a data retrieval method applicable to the data editing device of the present invention.

FIG. 6 shows a result of the retrieval method of FIG. 5.

FIG. 7A to FIG. 7D show a data retrieval screen used in the multiple physiological data collection and analysis device of the present invention.

FIG. 8 shows an example of the display content of a data retrieval result of the present invention.

FIG. 9 shows a flow chart of the method for analyzing multiple physiological data of the present invention.

FIG. 10 shows the flowchart of an embodiment of uncovering a new algorithm in the analysis of physiological data using the multiple physiological data collection and analysis device of the present invention.

FIG. 11 shows a waveform of the result of machine learning for various PSG detection results.

FIG. 12 shows a waveform of an analysis model obtained by machine learning using the invented multiple physiological data collection and analysis device.

FIG. 13 shows the correlation values resulted from a new algorithm uncovered by the invented multiple physiological data collection and analysis device.

### DETAILED DESCRIPTION OF THE INVENTION

Hereinafter, several preferred embodiments of the multiple physiological data collection and analysis device of the present invention will be described with reference to the drawings. It must be noted that the descriptions and illustrations of the embodiments of the present invention are only intended to present the main features and possible implementation modes of the present invention in a brief manner. The scope of the present invention should include implementations that can be derived of or deduced by the skilled persons in the industry.

Although it is not intended to limit the scope of the present invention, the inventors have found the various types of physiological data can be classified into four categories:
1. “Frame-featured” physiological data—This category of physiological data can be defined as “physiological data measured at a certain point in time, recorded in numerical values or images.” A set of frame-featured physiological data can be seen as a snapshot, which may be numbers or an image and so on, corresponding to an instance. Examples of this category include body height, body weight, blood pressure, blood glucose, body temperature, blood oxygen, X-ray photography, CT photography, room temperature, GPS location etc.
2. “Signal-featured” physiological data—This category of physiological data can be defined as “the continuous physiological data measured in a certain period of time, and expressed in a waveform encoded in the time domain (such as PCM) or frequency domain (such as SBC).” A “signal” extends for a continuous period on the time axis and can be seen as a video in terms of physiological signals. The most important features of “signals” are in their changes over time. Examples of physiological data of this category include: ECG, EEG, EMG, continuous blood oxygen, nasal airflow, chest and abdomen tension, heart rate, blood glucose, blood pressure. etc. readings.
3. “Multi-lead signal-featured” physiological data—This category of physiological data can be defined as “the continuous physiological data measured by a plurality of inspection devices synchronously in a certain period of time” Examples of this category include: multi-lead ECG, multi-lead EEG, multi-lead EMG etc.
4. “Multiple frame-featured” physiological data—This category of physiological data can be defined as “a combination of multiple frames defined by a first frame (I Frame) and differentiations (B Frame) thereto.”. Examples of this category include: Common multi-page information includes: videos, continuous CT photography etc.

Mainly based on the above findings and in combination with other unique technologies, the present invention provides a useful mechanism that can gather physiological data of different types, with different features, in different formats, and stored in different media and stored them in one single database, after suitable process, for retrieving, displaying, marking, processing them in a single interface, for further machine learning, deep learning and other processing.

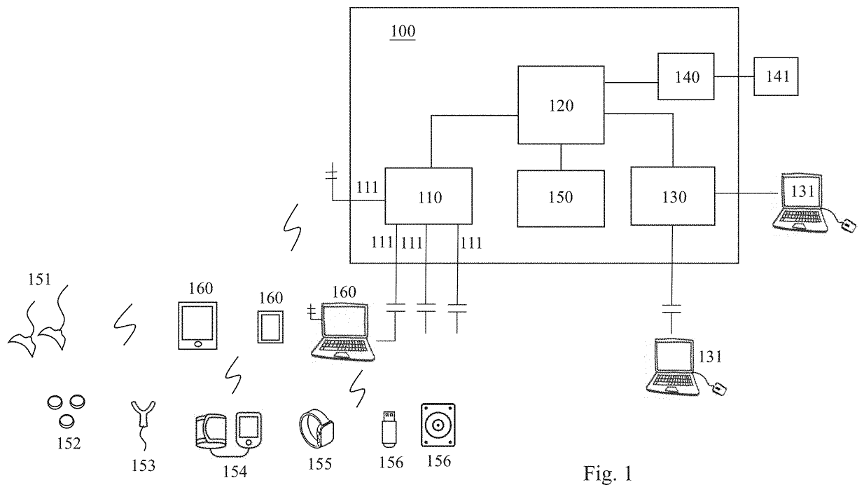

FIG. 1 is schematic diagram of an embodiment of the multiple physiological data collection and analysis device of the present invention. As shown in the figure, the multiple physiological data collection and analysis device 100 of the present invention can be implemented in a server computer, and can provide necessary data exchange, processing, storage, and display functions in an application program or in another applicable form. The multiple physiological data collection and analysis device 100 includes a data uploading device 110 that provides a variety of communication channels 111 for the communication connection of the plurality of physiological data sensing devices 151-155 or the physiological data storage device 156, so as to upload the physiological data detected by the physiological data sensing devices 151-155 or the physiological data stored in the physiological data storage device 156 to the multiple physiological data collection and analysis device 100. In the preferred embodiments of the present invention, the communication channel 111 is preferably the Internet. Specifically, the communication channel 111 is preferably a channel from the physiological data sensing device 151-155 or the physiological data storage device 156, via an mediation device 160, such as a smart phone or a tablet computer, to the multiple physiological data collection and analysis device via the Internet 100. The physiological data storage device 156 can also be connected to the multiple physiological data collection and analysis device 100 via, for example, a card reader, a USB interface, or a short-distance wireless communication channel.

The multiple physiological data may be one or more than one of the various types of physiological data of EEG, EMG, ECG, EOG, blood oxygen saturation (SaO2) and pulse, Tho/Abdo Effort, Nasal-oral air flow etc. Other information that can describe the situation of a human body, organs, tissues or a part or a combination thereof can also be applied to the present invention. The physiological data storage device 156 may be a storage device of any type, with any memory capacity, or connected in any way, such as cloud drives, external hard drives, USB memory cards, static hard drives, or even mobile phones, tablets. It may also be a laptop or desktop computer, or another server computer.

Almost all the instruments used to measure the above-mentioned physiological data available in the market have already provided Internet access capabilities. Otherwise, one can connect a sensing device to the Internet via such as a smartphone or a tablet through short-distance communication protocols such as Bluetooth to transmit the sensing results. In the prior art, installing an application program in a smart phone or tablet, or other computer devices with Internet access capabilities, i.e., the mediation device 160, to receive the physiological data from a variety of physiological data sensing devices 151-155, so that the mediation device 160 can provide the physiological data to the multiple physiological data collection and analysis device 100, is already a known technology. Detailed technology thereof is thus omitted.

The multiple physiological data collection and analysis device 100 provides a data storage device 120 in connection with the data upload device 110. The data storage device 120 provides a large volume of memory space to store the physiological data uploaded by the plurality of physiological data sensing devices 151-155 and the physiological data storage device 156. The data storage device 120 also provides a memory space to store the processing result data generated by the multiple physiological data collection and analysis device 100 after processing the stored or uploaded physiological data. The configuration of the data storage device 120 is an important technical feature of the present invention and its relevant details will be explained below.

The multiple physiological data collection and analysis device 100 further comprises a data editing device 130 that is connected to the data storage device 120 and provides a human-machine interface 131 for the user to retrieve specific physiological data and/or processing results from the data storage device 120, for browsing, manually marking or modification of markers. The human-machine interface 131 may include one or more of input/output devices such as a display device, a mouse, a keyboard, a microphone, and a loudspeaker, and may also include other tools that can add, delete, and change content in a physiological data file. The human-machine interface 131 of the data editing device 130 provides a retrieval tool for users to input indices to call out one or more physiological data sets that contain corresponding indices, and to display the physiological data on the human-machine interface 131 in a predetermined form and arrangements, for the users to edit. After the user finishes editing, the processing result can be indexed and stored in the data storage device 120.

According to a preferred embodiment of the present invention, the data storage device 120 provides an automatic indexing capability, which can automatically mark and index each set of the inspection result physiological data and/or processing result physiological data. In such embodiments, the data editing device 130 is configured to retrieve a physiological data set, in response to an indexed request of a user.

According to a preferred embodiment of the present invention, the data storage device 120 of the multiple physiological data collection and analysis device 100 stores the physiological data corresponding to a plurality of person. Each set of physiological data is indexed in the following way:
1. For “signal-featured” physiological data and “multi-lead signal-featured” physiological data: file name, recording time and an identification code (ID code).

In them,
1) File Name—Any file name that is able to identify the type of the physiological data. The file name may be given in the form of:

[Personal ID code+type of physiological signal+device brand+device model name]. Of course, other forms of a file name may also be used in the present invention.
2) Recording Time—The recording time can be a time point or a time period defined by a start time and an end time.
3) ID Code—It is preferably a unique code and is related to the numerical values in the corresponding physiological data file. The code length should be moderate; it should not be too short, so that it is easily coincided with other data set, and it should not be too long, which increases processing complexity, resources and time. In the preferred embodiment of the present invention, the hash value calculated from to the numerical values of the file content, especially the “Secure Hash Algorithm 256-bit” (SHA256) value is preferred. Due to the moderate length of this SHA256 code, there is almost no repetition, so it is particularly suitable for the present invention.
4) A suited example of the file name is given here:

UuIiDdd1234-ECG-LARGAN-AT202, in which,

UuIiDdd1234=User ID

ECG=ECG signal

LARGAN=equipment manufacturer

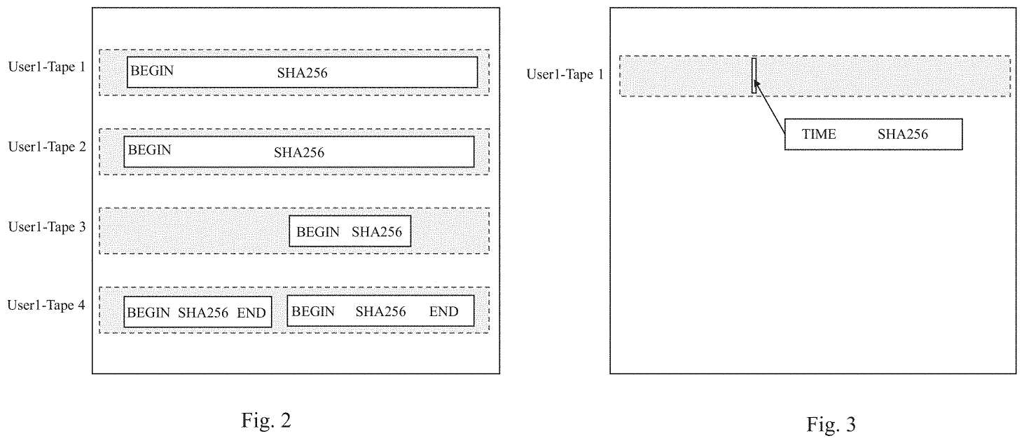

AT202=device model
5) File names can be encrypted to prevent leakage of sensitive data. FIG. 2 shows several archive formats useful for the signal-featured physiological data.
2. For “frame-featured” physiological data and “multiple frame-featured” physiological data: file name, recording time and an identification code (ID code).
   1) File Name—Any file name that is able to identify the type of the physiological data. The file name may be given in the form of:

[User ID+type of physiological signal+device brand+device model name].

Of course, other forms of a file name may also be used in the present invention.
2) Recording Time—The recording time is mostly a time point. For multiple sequential frames, only the recording time of the first frame (I Frame) needs to be identified.
3) ID Code—It is preferably a unique code and is preferably an SHA256 hash value.
4) A suited example of the file name is given here:

UuIiDdd1234-GLU-ABC-VP123, in which,

UuIiDdd1234=User ID

GLU=blood glucose level

ABC=equipment manufacturer

VP123=device model
5) File names can be encrypted to prevent leakage of sensitive data. FIG. 3 shows an example of the archive format useful for the frame-featured physiological data.

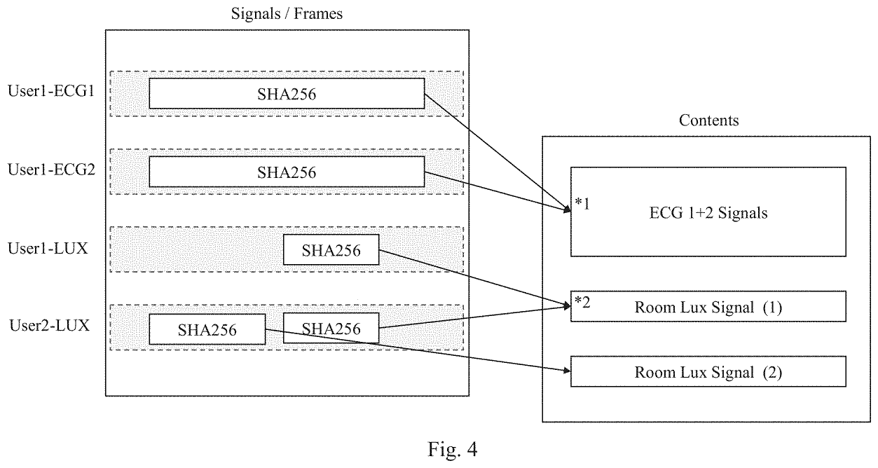

FIG. 4 shows a schematic diagram of a data structure for physiological data stored in the data storage device of the multiple physiological data collection and analysis device of the present invention. By assigning each set of the physiological data an index, a plurality of physiological data sets may be related on the person, the date/time, or other common features, thus can be retrieved and displayed on the same screen at the same time for browsing, comparison, searching for relevance, marking, and other processing such as machine learning and deep learning. The resulted data can also be used in the same or similar applications/processing.

In most preferred embodiments of the present invention, the hash function is chosen to calculate the ID code, mainly because the hash code is relatively short in length among all the indexing methods that are not prone to collision (different contents produce the same code value) and do not involve complicated calculations. In particular, the SHA256 code is only 256 bits long, therefore is highly suitable as a database index. In calculation, only bit reversal (XOR), shift (SHIFT), and rotation (ROT) are used; it is efficient and easy to implement. The advantages of using the SHA256 hash code as the index of a physiological data set include:
1. If data sets with the same contents were uploaded, only one copy of the data sets will be kept. Duplicated data sets can be easily found, because they would have the same hash code.
2. The hash code can also be used to determine whether the data are damaged or tampered with.
3. “Signal-featured” physiological data and “multi-lead signal-featured” physiological data can share content without taking up additional space.

The physiological data uploaded by the data uploading device 110 are processed as described above and then saved in the data storage device 120 for later use.

As described above, the data editing device 130 of the present invention is configured to determine the relevance of different sets of physiological data, in particular, based on the index of each data set, and display the multiple physiological data that are determined to have relevance as the retrieval result.

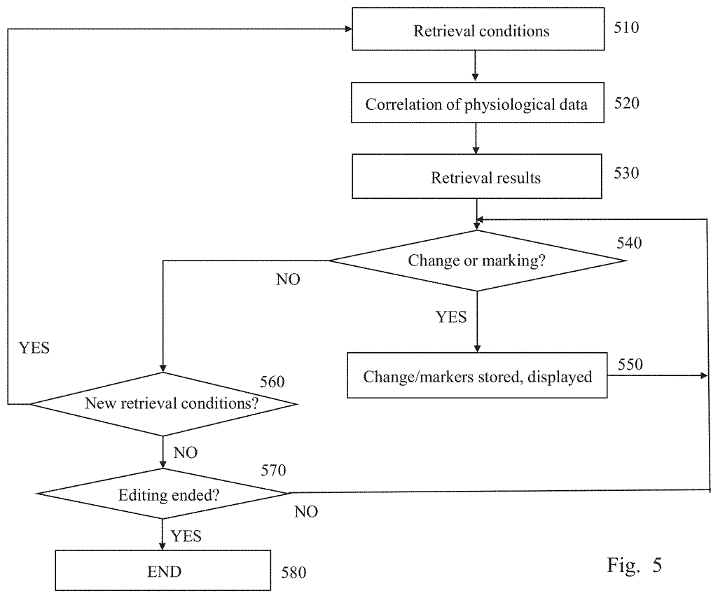

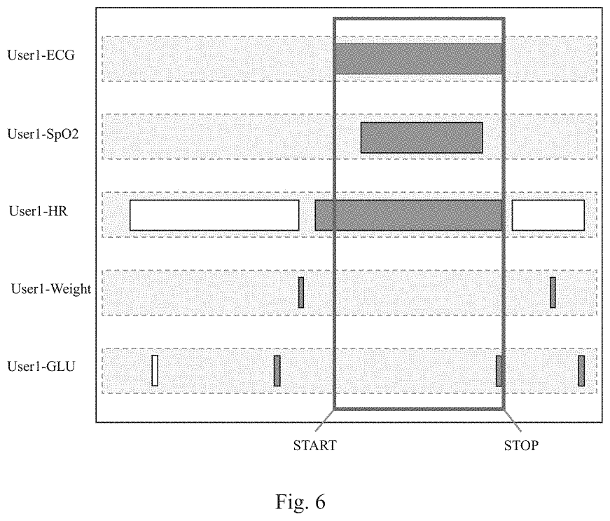

FIG. 5 shows a flow chart of a data retrieval method applicable to the data editing device 130 of the present invention. FIG. 6 shows a result of the retrieval method of FIG. 5. As shown in FIG. 5, in step 510, after the user inputs retrieval conditions on the human-machine interface 131 of the data editing device 130, the data editing device 130 starts data retrieval. In a general case, the retrieval conditions would include the personal information of the person from where the physiological data are, the date of collection, and the type of the data. In a preferred embodiment of the present invention, the data editing device 130 is configured to automatically retrieve the personal ID code corresponding to the personal information in the database, after receiving the input personal information. In response to the input retrieval conditions the data editing device 130 searches and retrieves all physiological data that satisfy the retrieval conditions from the data storage device 120. At step 520, the data editing device 130 determines possible correlations among the retrieved physiological data. For signal-featured physiological data, all physiological data that have common features are retrieved. On the other hand, for frame-featured physiological data only an optimal frame is retrieved.

In the foregoing steps, the correlation of two sets of data may be determined, when they have a time slot in common. For example, a plurality of sets of data whose recording time falls within a certain time period may be determined as correlated. Other methods that can determine the relevance based on the content of the data file, especially the relevance based on an element/component of the indices of a physiological data set, can also be applied to the present invention.

As for the best frame of frame-featured data, it usually refers to the data that the searcher is most likely interested. Therefore, it can also be determined based on its time feature. Other data content that can be determined as most suitable for display based on the content of the data file, especially based on the components of the indices, can also be determined as the best frame.

Specifically, the method for describing the frame-featured physiological data and the signal-featured physiological data is different. The frame-featured physiological data need to describe a value, and to define its dimension and precision (resolution). The signal-featured physiological data on the other hand adds a description of the sampling rate and the filtering method, and requires more attention on the dynamic range of changes. The multiple frame-featured physiological data and the multi-lead signal-featured physiological data are essentially frame-featured physiological data and signal-featured physiological data, respectively, provided, however, that the data included therein cannot usually be recorded and read separately. They are configured into multiple/multi-lead, mainly to facilitate simultaneous access and recording. For example, the ECG signal of 5 leads usually needs to be viewed in parallel at the same time. It is not meaningful to look at a lead alone. Dividing it into 5 independent data sets during recording would simply lead to low efficiency.

The frame-featured physiological data and the signal-featured physiological data are different in data processing and use. The frame-featured physiological data are only a point in time. Although the values of a set of data outside this time point are unknown, the values can be estimated from the values measured beforehand and afterward. For example, if there is only one white spot on the chest X-ray taken a year ago, and there is only one white spot on the chest X-ray taken today, it can be presumed that in all chest X-ray taken in the past year there should be only one white spot.

On the other hand, the signal-featured physiological data occupy a continuous section on the time axis. Only the measured values of an approximate time section or an intersection can be used as reference. For example, a patient wears an oximeter from 20:00 last night to 5:00 this morning. If his/her sleep disordered breathing index for from 22:00 to 8:00 needs be analyzed, the blood oxygen readings of the intersection between 22:00 and 5:00 can be used.

Many times, people want to find a causal relationship between a signal and a frame. For example, when a specific event (frame) occurs, people want to know if it will be accompanied by a continuous signal (signal) with specific symptoms. A good example is, experts have discovered through observation that when a sleep apnea event occurs, the heart rate will first decrease and then increase. As long as the heart rate is monitored for signs of first decreasing and then increasing, it can be used to assess whether a sleep apnea event has occurred. In this respect, the present invention can provide useful information. Through machine learning, it is possible to discover the correlation between heart rate changes and sleep apnea events. After verification, new analysis methods can be discovered.

Then, in step 530, the data editing device 130 displays the retrieved data on the human-machine interface 131 in a predetermined format. The form of display is usually images, especially graphics. However, other forms of data display, such as text, sound, animation, continuous images or discontinuous images, are also applicable.

In step 540, the data editing device 130 determines whether the user has marked or modified a manual marker. If YES, in step 550, the changes made by the user is stored in a data file that is the same as or different from the corresponding data file being displayed, and the displayed content is changed accordingly. The step returns to 540. If the judgment result of step 540 is NO, then it is determined in step 560 whether new retrieval condition are input. If YES, the step returns to 510; otherwise, it is determined in step 570 whether to end the editing. If NOT, the step returns to 540; otherwise, the editing ends in step 580. In the above steps, researchers can easily discover a possible relation between/among various types of physiological data and/or the correlation of a type of physiological data and specific physiological phenomena from the displayed information.

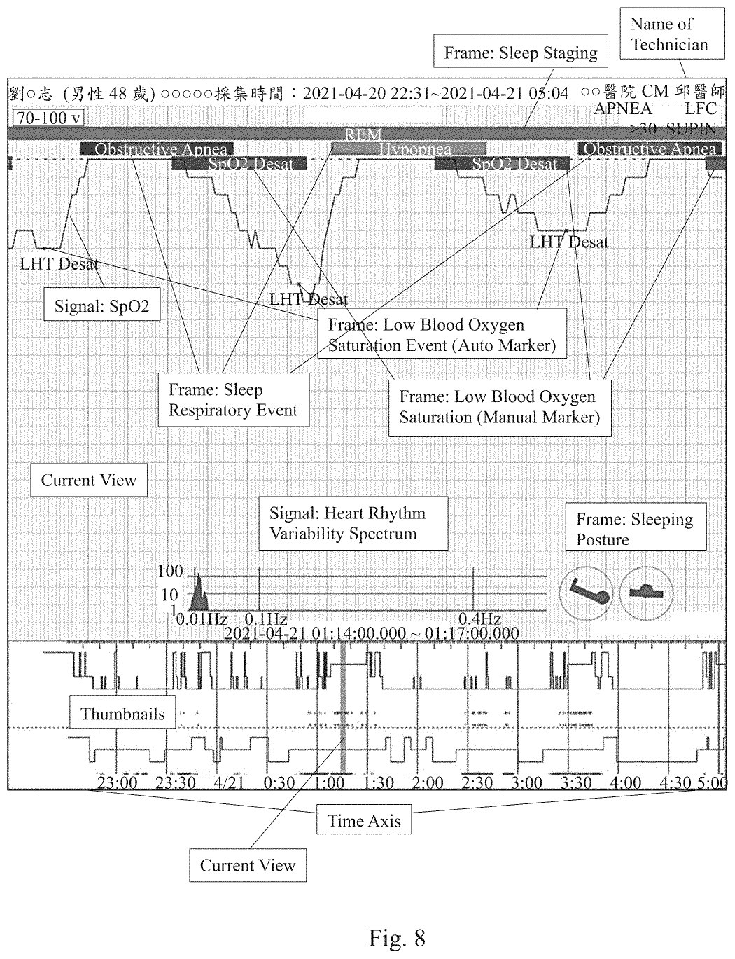

In terms of application, when researchers find physiological phenomena that arouse interest, they can mark manual markers on them. The manual markers can be an icon or a string of words. The data editing device 130 automatically attaches the manual markers to the physiological data file for future use. FIG. 8 shows some examples of the manual markers applicable in this invention.

The retrieval result in FIG. 6 shows that the physiological data detected by different instruments can be displayed on the same display device at the same time. Information of different nature and forms can also be displayed altogether according to their relevance, such as relevance in time, for easy to compare and determine. The physiological data of different people can also be displayed together.

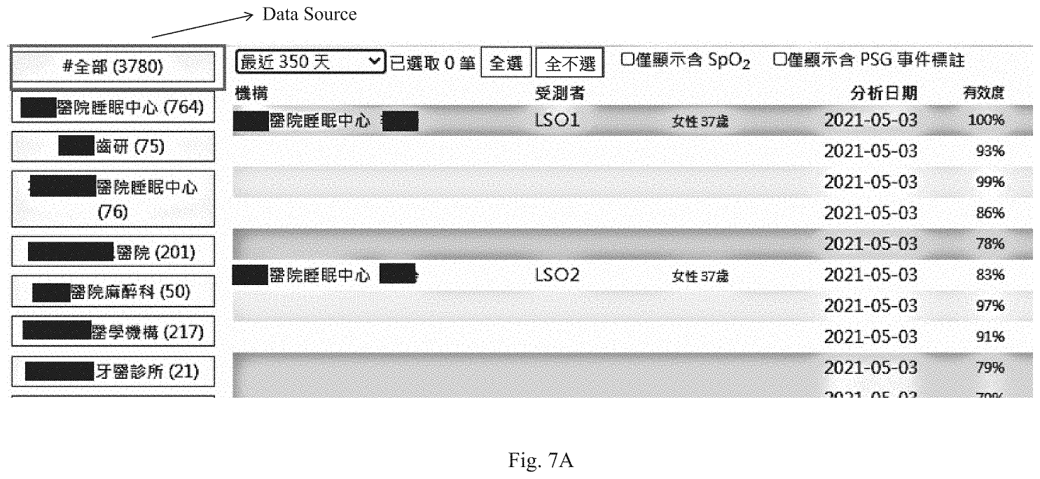

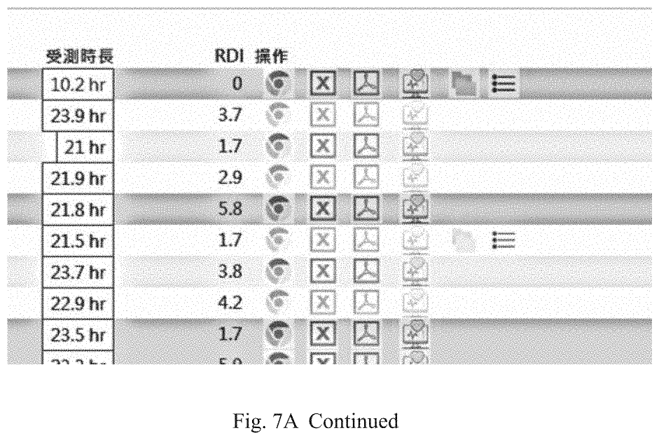

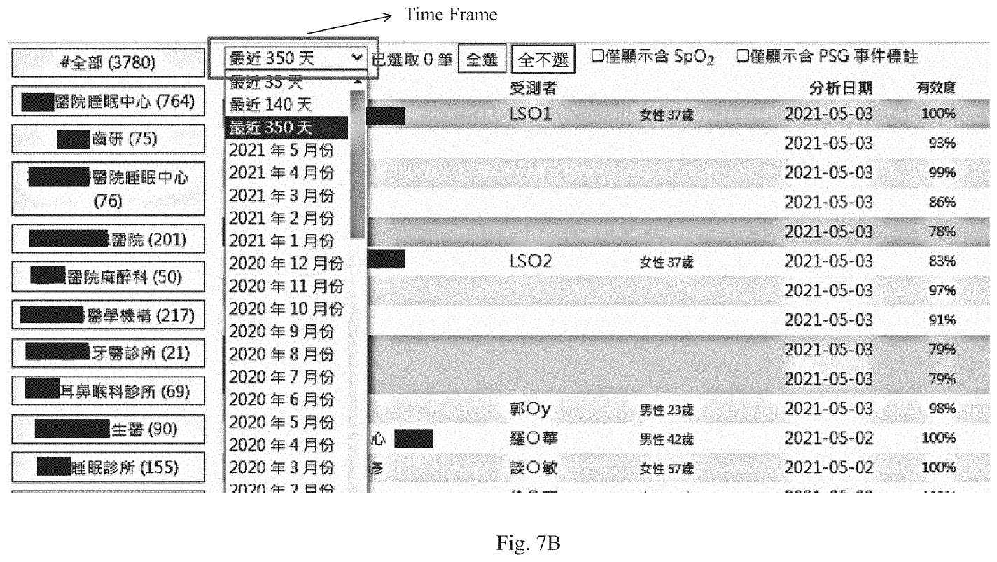

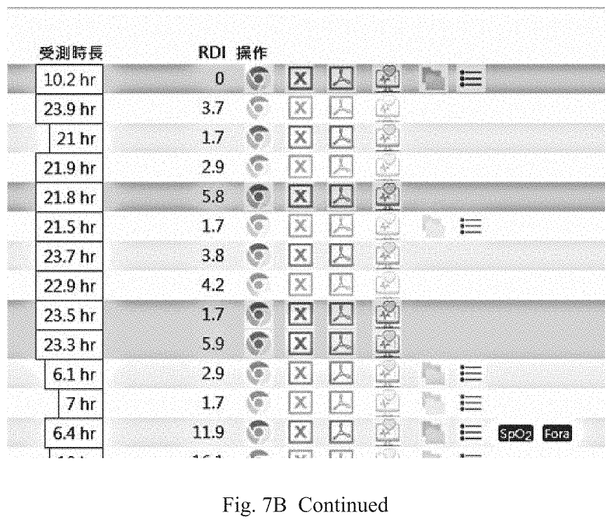

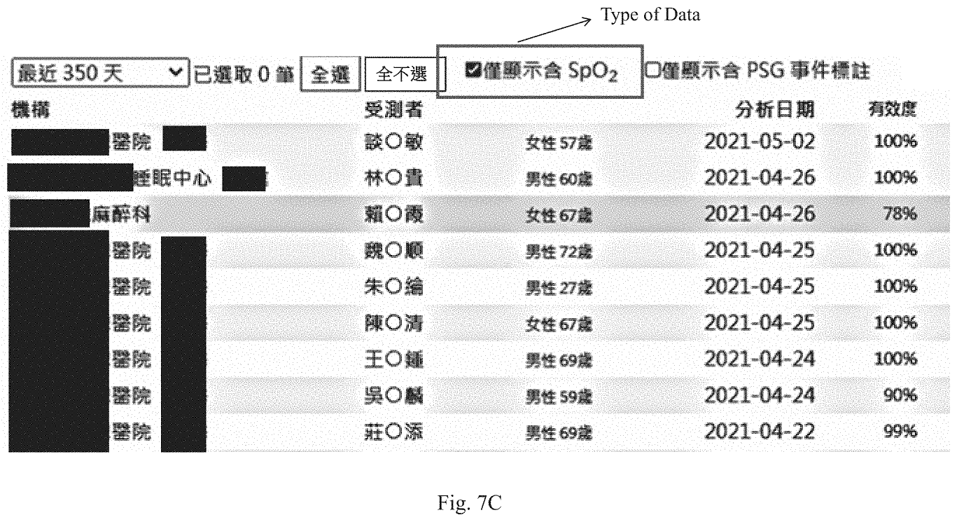

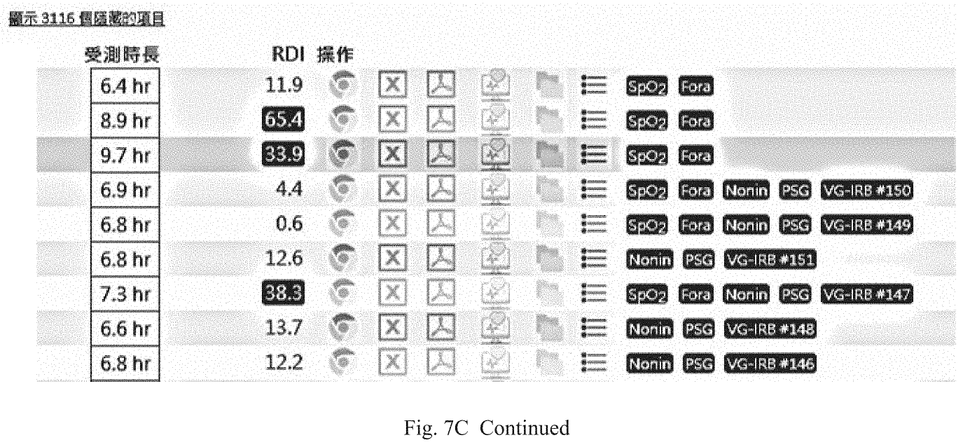

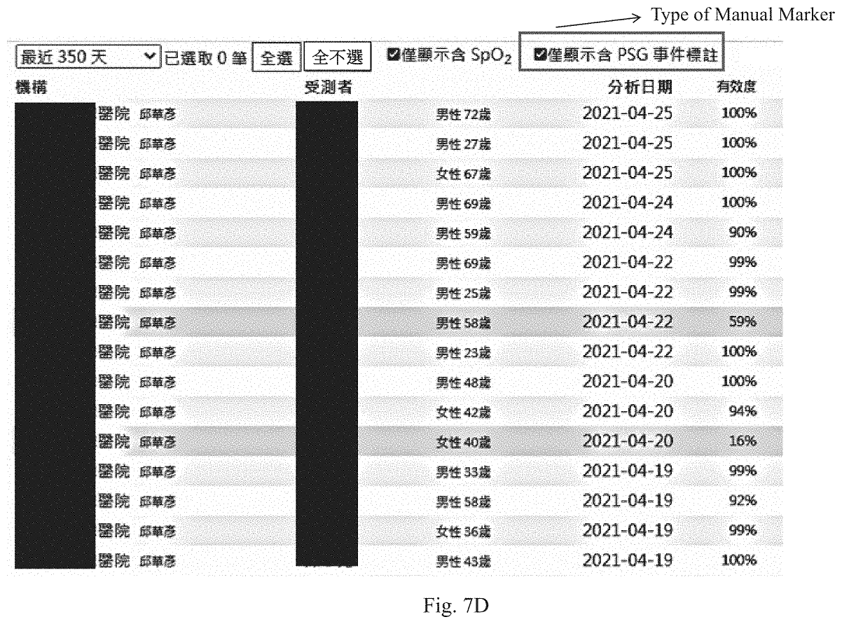

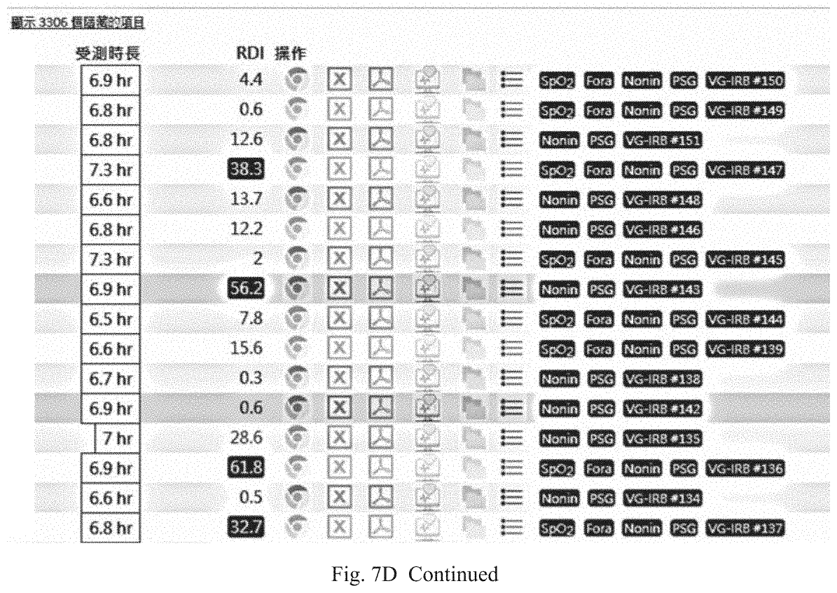

FIG. 7A to FIG. 7D show one example of the data retrieval screen used in the multiple physiological data collection and analysis device of the present invention. 7A shows one page displayed on the human-machine interface 131 of the data editing device 130. In this example, entry fields for the following retrieval conditions are provided in the function column of the retrieval page:
1. Data source: The name of the institution that provides the specific multiple physiological data, such as the name of a hospital/clinic, a sleep center, etc. FIG. 7A shows the search results after selecting two of the medical institutions.
2. Time frame of data collection: The dates, time frame of the collection of the multiple physiological data. The field may automatically generate the starting and end dates. FIG. 7B shows the search results after selecting a specific time period.

3. Type of data: The type of the data, such as EEG, EMG, ECG, EOG, SaO2 and pulse, Tho/Abdo Effort and Nasal-oral Air Flow. Other types of physiological data, or even other categorization methods, can also be applied to the present invention. FIG. 7C shows the search result of selecting “SpO2 only.”
4. Manual markers: The markers made by a professional on the physiological data using the data editing device 130. Generally speaking, the markers made by professionals need to specify standard terminology for correct retrieval. This, however, is not any technical limitation. FIG. 7D shows the search result of selecting “only PSG events”.

It can be seen from FIG. 7D that the present invention provides a very useful tool to retrieve relevant physiological data and to display them on the same screen. What's more special is that, in addition to displaying data collected for a specific person and time, the displayed items can also include physiological data for different people, measured on different dates, and on different numerical distribution ranges, as well as in various forms, types, and natures. The various forms, types, and properties of the physiological data can be expressed with different icons and/or in different colors, in order to let users to know the approximate distribution of the search results at a glance.

FIG. 8 shows an example of the display content of a data retrieval result of the present invention. Shown in FIG. 8 are sets of physiological data that represents the blood oxygen concentration signal (Signal: SpO2) detected by a specific person during a specific period of time, and the manual markers assigned by an interpretation expert in the physiological data (Frame: Sleep Respiratory Event & Sleep Staging). The retrieved information can be displayed in a graphic manner. In addition, time thumbnails are also used in the graph, so that users can immediately understand the exact test time. In addition, the heart rhythm variability spectrum is presented in the form of signal-featured data, while the sleeping posture is presented in the form of frame-featured data. The contents displayed here include multiple physiological data and manual makers added by experts, which are easy to understand and their relevance can easily catch attention.

The multiple physiological data collection and analysis device 100 of the present invention may also include an automatic analysis device 140. The automatic analysis device 140 provides a filtering function and receives a filtering command from a user through the filtering interface 141, to retrieve from the data storage device 120 physiological data and/or processing result physiological data corresponding to a filtering condition included in the filter command. The filtering result data are useful for machine learning, in discovering algorithms that can be executed by a computer system, or for AI deep learning, to find out a type of physiological data that is correlated to a manual marker, i.e., a physiological phenomenon. Researchers can provide the filtering results to a machine learning program, and use approaches such as try-and-error to find out an algorithm that can be interpreted by the machine. Researchers can also provide the filtering results to an AI deep learning program, to find out a type of physiological data that is related to a manual marker.

The analysis techniques suitable for the automatic analysis device 140 of the present invention include various deep learning techniques. Existing deep learning technologies can already assist in finding from a database containing a large quantity of data specific types of physiological data that may be related to specific physiological phenomena. For example, Sun et al. proposes a methodology applicable to the present invention. See Haoqi Sun et al., Sleep staging from electrocardiography and respiration with deep learning, Sleep staging from electrocardiography and respiration with deep learning, https://pubmed.ncbi.nlm.nih.gov/31863111/). Other experts in this technical field have also proposed several technologies that can be applied in the present invention, which are all included herein for reference.

What is notable is, since the present invention has provided a simple and graphical interface that can display different types of physiological data and specific physiological phenomena on the same screen, the relevance of a type of the physiological data and certain manual markers can easily catch the attention of an observer. Researchers only need to try multiple times to retrieve different combinations of physiological data and verify their correlation with certain physiological phenomena (markers). It is possible to find a connection between certain types of physiological data and physiological phenomena that was unknown before. In other words, the human-machine interface provided by the data editing device 130 of the present invention is a tool that makes it easy for researchers to see the correlation between certain types of physiological data and certain physiological phenomena with their naked eye. With manual selection of samples, researchers may discover new algorithms for monitoring, diagnoses, treatment and/or improvements of bodily disorders.

The multiple physiological data collection and analysis device 100 of the present invention provides a correlation evaluation device 150 for calculating the correlation value of a specific type of physiological data and a specific manual marker. After the user inputs a type or a combination of types of physiological data of the filtering results in the filtering interface 141, the correlation evaluation device 150 retrieves a specific range of physiological data from the data storage device 120, and calculates a correlation value of the type of physiological data and a manual marker that was input by the user also in the filtering interface 141. The evaluation results are then displayed in a numerical or graphical form.

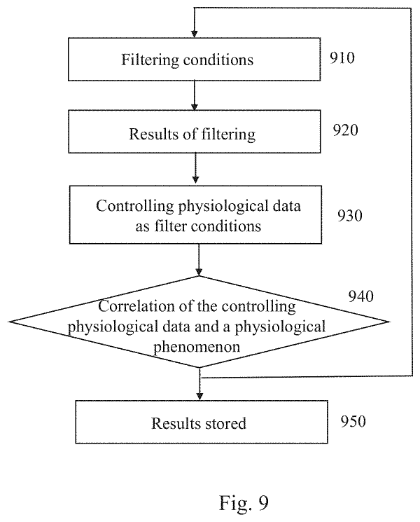

FIG. 9 shows a flow chart of the method for analyzing multiple physiological data of the present invention. As shown in the figure, in step 910, the user enters certain filtering conditions on the filtering interface 141. The applicable filtering conditions may be specific manual markers. Taking the study of sleep-respiratory event as an example, possible filtering conditions may be physiological data marked with sleep-respiratory events (Apnea, Hypopnea, Desat). However, since the purpose of machine learning is to find unknown analysis methods, the filtering conditions can also be random conditions, such as the average distribution of age and gender. In addition, the filtering condition can also be an exclusion condition, for example, physiological data marked with sleep respiratory events, but excluding data manually marked as “arrhythmia (VPC, APC, AF, AFib).”

In step 920, the automatic analysis device 140 displays the filtering results on the filtering interface 141. In this step, the automatic analysis device 140 may provide the user with the following filtering functions:
1. Choose a suitable training model (CNN, RNN, LSTM, ReLU, etc.)
2. Define the output layer (For a sleep breathing event, the output may be “with/without” obstruction).
3. Define the input layer (permutation and combination of different signal-featured/frame-featured physiological data).
4. Arrange the samples by input layer and input them into the training model in order.
5. Find the input layer with the smallest error to generate the controlling physiological data combination

The above filtering conditions are not in a certain order. It's acceptable to omit or add one or more filtering conditions. What is important is to find the right amount of relevant physiological data to save time in machine learning or deep learning.

In step 930, the user inputs the controlling physiological data of the filtering result into the filtering interface 141. In step 940, the correlation evaluation device 140 generates a result, which may be a presumed relevance of a controlling physiological data and a physiological phenomenon. The correlation value of the two is then evaluated. The controlling physiological data may include signal-feature and frame-featured physiological data, while the physiological phenomenon is usually a disease or a physiological abnormality. If the evaluation result is “highly correlated,” it means the finding is successful, and the result is stored in step 950. A new analysis is added or updated to the multiple physiological data collection and analysis device 100. Otherwise, the step returns to 930 or 910 for further filtering.

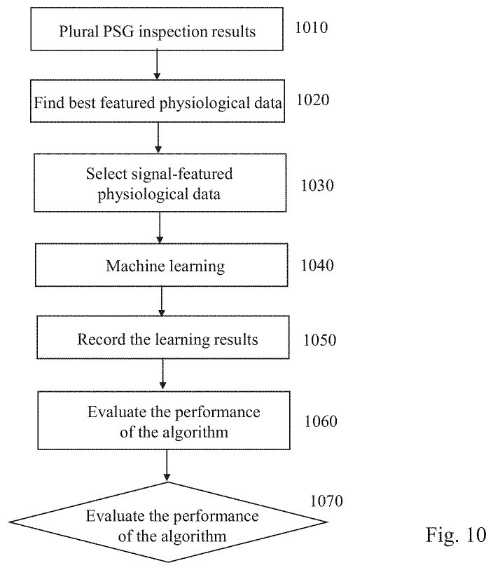

In the followings, specific examples are used to illustrate how researchers use the invented multiple physiological data collection and analysis device to discover and verify the correlation of a specific type of physiological data and specific physiological phenomena. In this embodiment, the readings obtained by PSG (Multiple Physiological Examination of Sleep) are used as entry, for the invented multiple physiological data collection and analysis device to perform machine learning in an attempt to discover and establish a new algorithm for sleep staging and sleep respiratory analyses. FIG. 10 shows the flowchart of an embodiment of discovering a new algorithm in the analysis of physiological data using the multiple physiological data collection and analysis device of the present invention.

As shown in the figure, in step 1010, various PSG detection results are input into the system in the form of Signal (signal-featured) data and Frame (frame-featured type) data for machine learning and evaluation. The signal-featured physiological data used in this example include: EEG, EMG, ECG, EOG, blood oxygen saturation (SaO2) and pulse, Tho/Abdo Effort, Nasal-oral air flow, microphone voice, body movement, leg movement etc. As for frame-featured physiological data, they include records such as manual markers for sleep staging and manual markets for respiration events. Among them, the relevant manual markers are those marked by professionals in the relevant physiological data files using the multiple physiological data collection and analysis device of the present invention.

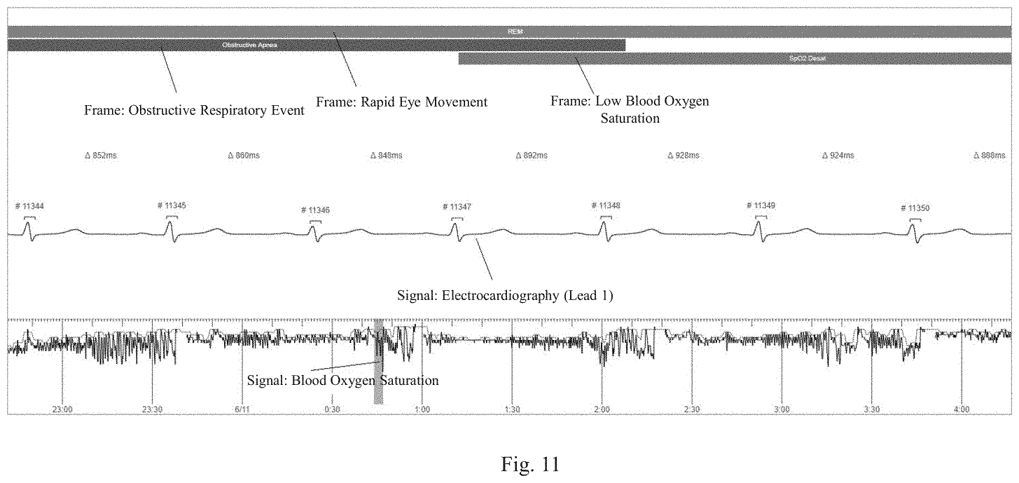

FIG. 11 shows a waveform of the result of machine learning for various PSG detection results in step 1010. The chart shows that the recorded results contain a variety of physiological data, arranged according to their indices, indicating the possible relevance among them.

In step 1020, one or several types of signal-featured physiological data with higher correlation values to the manual markers are found. In this step, it is preferable to use an AI-equipped computer to execute a deep learning algorithm to find the best features in the above-mentioned signal-featured physiological data relative to a specific manual marker, followed by sorting the correlation values according to a recognizability of the best features against the manual markers. In this example, it is easy for any user to identify the following possible correlations from the displayed results:

For sleep staging related manual markers, the types of physiological data are arranged in descending recognizability order as: EEG>ECG>Snout and nose airflow>Chest movements> . . . .

For respiration event related manual markers, the types of physiological data are arranged in descending recognizability order as: blood oxygen>oral and nasal air flow>ECG>chest undulation> . . . .

In more detail, taking the ECG as an example, the best feature for sleep staging is heart rate variability (time domain). The best feature for sleep breathing events is heart rate variability (frequency domain). In this way, one or several possible combinations can be presumed, that is, the correlation of specific types of physiological data, or even certain parameters thereof, and specific manual markers can be established or presumed.

In this embodiment, the recognizability may be quantized as a correlation value, which in turn can be determined by an AUC value. If the AUC value is used to represent the recognizability, the closer the value is to 1, the better. If the value is below 0.6, the correlation is considered insufficient and the type of physiological data is not selected as a controlling feature. As for the parameters selected for specific types of physiological data in verifying their correlation values, they can be selected by first referring to the suggestions mentioned in the literature. The system of the present invention can then use deep learning to verify the correlation value of the parameters and to find useful parameters not mentioned in the literature.

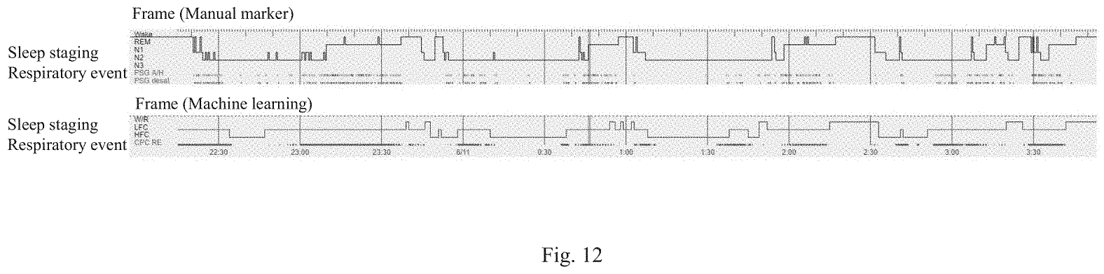

Next, in step 1030, a type of signal-featured physiological data for entry is selected. As mentioned above, the selection method is preferably manual selection. In this example, considering the convenience of the user's operation and the relevance to the previous steps, two types of data such as blood oxygen and ECG can be selected as entries. The main reason for choosing ECG instead of EEG in this example is that EEG has poor recognizability for sleep breathing events. At the same time, there are more electrodes for measuring EEG. The subjects cannot stick it on their own, and they tend to fall off during sleep. On the other hand, EEG is only suitable for use in situations where someone is supervised by others. In the context of home measurement, ECG is preferred. In step 1040, machine learning is performed using the features found in step 102 to train a machine learning model. In step 1050, the result of the machine learning is recorded. In a preferred embodiment, it can be recorded in the form of frame-featured physiological data. FIG. 12 shows a waveform of an analysis model obtained by machine learning using the invented multiple physiological data collection and analysis device.

In step 1060, the performance of the algorithm so found is evaluated. Compare various indicators of manually marked frame-featured physiological data with machine algorithm marked frame-featured physiological data:

Sample distribution: Statistical sample distribution (gender, respiratory problem degree) from the frame-featured training samples, with the results obtained as follows. It is determined that the samples used in this embodiment are representative:

Sample Distribution

| | | | | | |
|---|---|---|---|---|---|
| Non-OSA | 150 | 27% | | | |
| Mild OSA | 113 | 20% | | | |
| Moderate OSA | 104 | 19% | Female | 112 | 20% |
| Severe OSA | 186 | 34% | Male | 441 | 80% |
| Total | 553 | 100% | Total | 553 | 100% |

Sensitivity and specificity: Compare the correlation value of the machine learning markers to the manual markers under different respiratory disorder indices (Apnea-Hypopnea Index, AHI) (AUC=accuracy, TP=true positive, FN=false negative, FP=false positive, TN=true negative). The results obtained are as follows:

Diagnostic accuracy of LHT RDI compared with PSG-AHI

| Group-based PSG AHI | AUC | TP | FN | FP | TN | Sensitivity | Specitivity |
|---|---|---|---|---|---|---|---|
| AHI < 5 vs AHI ≥ 5 | 0.924 | 365 | 38 | 33 | 117 | 90.6% | 78.0% |
| AHI < 15 vs AHI ≥ 15 | 0.930 | 258 | 32 | 52 | 211 | 89.0% | 80.2% |
| AHI < 30 vs AHI ≥ 30 | 0.959 | 167 | 19 | 41 | 326 | 89.8% | 88.8% |

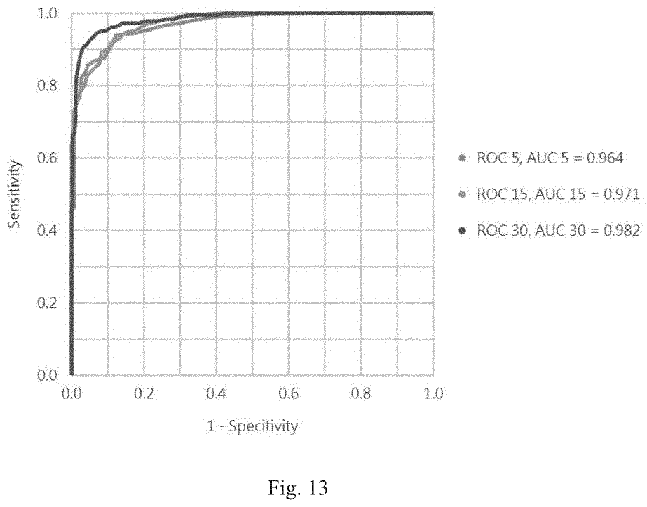

In step 1070, the correlation value of the obtained analysis method is used to judge whether the found algorithm is useful. The calculation result can be expressed numerically or graphically. For example, FIG. 13 shows the correlation values resulted from a new algorithm discovered by the invented multiple physiological data collection and analysis device, showing the coordinates of sensitivity and specificity. Users can more clearly judge from the chart whether the research results are useful. The method of judgment includes calculating the area under the correlation curve. Area>0.9 indicates excellent correlation. As shown in FIG. 13, the capability of revealing the recognizability of certain types of physiological data against specific manual markers of the analysis method generated by machine learning has been proven.

It should be understood that processes and techniques described herein are not inherently related to any particular apparatus and may be implemented by any suitable combination of components. Further, various types of general purpose devices may be used in accordance with the teachings described herein. The present invention has been described in relation to particular examples, which are intended in all respects to be illustrative rather than restrictive. Those skilled in the art will appreciate that many different combinations will be suitable for practicing the present invention.

Moreover, other implementations of the invention will be apparent to those skilled in the art from consideration of the specification and practice of the invention disclosed herein. Various aspects and/or components of the described embodiments may be used singly or in any combination. It is intended that the specification and examples be considered as exemplary only, with a true scope and spirit of the invention being indicated by the following claims.

What is claimed is:

1. A multiple physiological data collection and analysis device, comprising:
a data upload device to provide a communication channel for communication link of a plurality of physiological data sensing devices or physiological data storage devices, to receive different types of physiological data from the plurality of physiological data sensing devices or physiological data storage devices;
a data storage device to provide a memory space for storing various physiological data;
a data editing device to provide a human-machine interface for users to retrieve specific types of physiological data from the data storage device, and for browsing and for manually adding, deleting, or modifying a marker on a set of physiological data, and for user to select a type of physiological data as entry for determining a correlation of the selected type of physiological data and a manual marker; and
a correlation evaluation device to calculate a correlation value of the type of physiological data and a manual marker;
wherein the data storage device provides automatic indexing capability, to automatically index a set of physiological data, and wherein the data editing device is configured to display physiological data in an arrangement according to a corresponding index in response to an input request.

2. The multiple physiological data collection and analysis device according to claim 1, further comprising an automatic analysis device that provides a filtering interface to receive a filtering condition, automatically retrieves physiological data corresponding to the filtering conditions from the data storage device, and finds from the retrieved data a type of physiological data that is correlated to a specific manual marker.

3. The multiple physiological data collection and analysis device according to claim 1, wherein the physiological data stored in the data storage device correspond to a plurality of person.

4. The multiple physiological data collection and analysis device according to claim 1, wherein each set of physiological data is indexed with the following features:
for “signal-featured” physiological data and “multi-lead signal-featured” physiological data: file name, recording time and an identification code (ID code) and
for “frame-featured” physiological data and “multiple frame-featured” physiological data: file name, recording time and an identification code (ID code).
wherein the “signal-featured” physiological data are defined as: “continuous physiological data measured in a certain period of time, and expressed in a waveform encoded in the time domain (such as PCM) or frequency domain (such as SBC);”
the “multi-lead signal-featured” physiological data are defined as: “continuous physiological data measured by a plurality of inspection devices synchronously in a certain period of time;”
the “frame-featured” physiological data are defined as: “physiological data measured at a certain point in time, recorded in numerical values or images;” and
the “multiple frame-featured” physiological data are defined as: “a combination of multiple frames defined by a first frame (I Frame) and differentiations (B Frame) thereto.”

5. The multiple physiological data collection and analysis device according to claim 1, wherein a calculation result of the correlation evaluation device is displayed in a numerical value or a graph.

6. The multiple physiological data collection and analysis device according to claim 5, wherein the correlation evaluation result is determined according to a ratio of an area in the graph representing the calculation result of the correlation evaluation device.

7. The multiple physiological data collection and analysis device according to claim 5, wherein the calculation result of the correlation evaluation device is an AUC value, displayed in a numerical value or a graph.

8. The multiple physiological data collection and analysis device according to claim 1, wherein the correlation evaluation device further calculates a non-correlation value of a type of physiological data and a manual marker.

9. The multiple physiological data collection and analysis device according to claim 4, wherein the signal-featured physiological data comprises at least one selected from the group consisted of the following signals: ECG, EEG, EMG, continuous blood oxygen, nasal airflow, chest and abdomen tension, continuous heart rate, continuous blood sugar, continuous blood pressure.

10. The multiple physiological data collection and analysis device according to claim 4, wherein the multi-lead sign-featured physiological data comprises at least one selected from the group consisted of the following signals: multi-lead ECG, multi-lead EEG, and multi-lead EMG.

11. The multiple physiological data collection and analysis device according to claim 4, wherein the frame-featured physiological data comprises at least one selected from the group consisted of the following signals: height, weight, blood pressure, blood sugar, body temperature, blood oxygen, X-ray photography, CT Photography, room temperature, GPS location.

12. The multiple physiological data collection and analysis device according to claim 4, wherein the multiple frame-featured physiological data comprises at least one selected from the group consisted of the following signals: video and continuous CT images.

13. The multiple physiological data collection and analysis device according to claim 4, wherein the recording time comprises a time point or a time period defined by a start time and an end time.

14. The multiple physiological data collection and analysis device according to claim 4, wherein the ID code is related to a numerical value of the physiological data.

15. The multiple physiological data collection and analysis device according to claim 14, wherein the ID code comprises a hash value calculated according to the numerical value of the physiological data of a corresponding data set.

16. The multiple physiological data collection and analysis device according to claim 14, wherein the ID code comprises an SHA256 hash value calculated according to the numerical value of the physiological data of a corresponding data set.

17. The multiple physiological data collection and analysis device according to claim 2, wherein each set of physiological data is indexed with the following features:
for “signal-featured” physiological data and “multi-lead signal-featured” physiological data: file name, recording time and an identification code (ID code) and
for “frame-featured” physiological data and “multiple frame-featured” physiological data: file name, recording time and an identification code (ID code).
wherein the “signal-featured” physiological data are defined as: “continuous physiological data measured in a certain period of time, and expressed in a waveform encoded in the time domain (such as PCM) or frequency domain (such as SBC);”
the “multi-lead signal-featured” physiological data are defined as: “continuous physiological data measured by a plurality of inspection devices synchronously in a certain period of time;”
the “frame-featured” physiological data are defined as: “physiological data measured at a certain point in time, recorded in numerical values or images;” and
the “multiple frame-featured” physiological data are defined as: “a combination of multiple frames defined by a first frame (I Frame) and differentiations (B Frame) thereto.”

18. The multiple physiological data collection and analysis device according to claim 17, wherein a calculation result of the correlation evaluation device is displayed in a numerical value or a graph.

19. The multiple physiological data collection and analysis device according to claim 18, wherein the correlation evaluation result is determined according to a ratio of an area in the graph representing the calculation result of the correlation evaluation device.

20. The multiple physiological data collection and analysis device according to claim 18, wherein the calculation result of the correlation evaluation device is an AUC value, displayed in a numerical value or a graph.

21. The multiple physiological data collection and analysis device according to claim 17, wherein the correlation evaluation device further calculates a non-correlation value of a type of physiological data and a manual marker.

22. The multiple physiological data collection and analysis device according to claim 17, wherein the signal-featured physiological data comprises at least one selected from the group consisted of the following signals: ECG, EEG, EMG, continuous blood oxygen, nasal airflow, chest and abdomen tension, continuous heart rate, continuous blood sugar, continuous blood pressure.

23. The multiple physiological data collection and analysis device according to claim 17, wherein the multi-lead sign-featured physiological data comprises at least one selected from the group consisted of the following signals: multi-lead ECG, multi-lead EEG, and multi-lead EMG.

24. The multiple physiological data collection and analysis device according to claim 17, wherein the frame-featured physiological data comprises at least one selected from the group consisted of the following signals: height, weight, blood pressure, blood sugar, body temperature, blood oxygen, X-ray photography, CT Photography, room temperature, GPS location.

25. The multiple physiological data collection and analysis device according to claim 17, wherein the multiple frame-featured physiological data comprises at least one selected from the group consisted of the following signals: video and continuous CT images.

26. The multiple physiological data collection and analysis device according to claim 17, wherein the recording time comprises a time point or a time period defined by a start time and an end time.

27. The multiple physiological data collection and analysis device according to claim 17, wherein the ID code is related to a numerical value of the physiological data.

28. The multiple physiological data collection and analysis device according to claim 27, wherein the ID code comprises a hash value calculated according to the numerical value of the physiological data of a corresponding data set.

29. The multiple physiological data collection and analysis device according to claim 27, wherein the ID code comprises an SHA256 hash value calculated according to the numerical value of the physiological data of a corresponding data set.
